(19) United States
(12) Patent Application Publication
(10) Pub. No.: US 2022/0015748 A1
(43) Pub. Date: Jan. 20, 2022

(54) SYSTEMS AND METHODS FOR MINIMALLY INVASIVE DELIVERY AND IN VIVO CREATION OF BIOMATERIAL STRUCTURES

(71) Applicant: Nextern Innovation, LLC, White Bear Lake, MN (US)

(72) Inventors: John Swoyer, Blaine, MN (US); Steven M. Gigl, Crystal, MN (US); Richard Farrell, Delwood, MN (US)

(73) Assignee: Nextern Innovation, LLC, White Bear Lake, MN (US)

(21) Appl. No.: 17/305,856

(22) Filed: Jul. 15, 2021

Related U.S. Application Data
(60) Provisional application No. 63/154,192, filed on Feb. 26, 2021.
(60) Provisional application No. 63/053,197, filed on Jul. 17, 2020.

(51) Int. Cl. A61B 17/00; A61L 27/36; A61L 27/54

(52) U.S. Cl. CPC A61B 17/0057; A61B 17/00234; A61L 27/36; A61B 2017/003; A61B 2017/00623; A61L 2400/06; A61L 27/54

(57) ABSTRACT

Apparatus and associated methods relate to closure of a stoma with a structure continuously formed in vivo. In an illustrative example, a stoma closure tool (SCT) may include a drive module, a phase transition inducement module (PTIM), and a conduit that defines a lumen. A distal end of the conduit may, for example, be inserted through a first tissue and into a second tissue that together at least partially define a stoma. A flow rate of a fluid biomaterial through the lumen and discharged at the distal end of the conduit may, for example, be controlled by the drive module. A fluid to solid phase transition in the biomaterial may, for example, be controllably induced by the PTIM. Various embodiments may, for example, advantageously form a continuous structure extending directly across the stoma between a proximal anchor in the first tissue and a distal anchor in the second tissue.

### CROSS-REFERENCE TO RELATED APPLICATIONS

This application claims the benefit of U.S. Provisional Application Ser. No. 63/053,197, titled “SYSTEMS AND METHODS FOR MINIMALLY INVASIVE DELIVERY AND IN VIVO CREATION OF BIOMATERIAL STRUCTURES,” filed by John Swoyer, et al., on Jul. 17, 2020.

This application also claims the benefit of U.S. Provisional Application Ser. No. 63/154,192, titled “STEERABLE SHEATH WITH ROBOTIC HANDLE STAND,” filed by John Swoyer, et al., on Feb. 26, 2021.

This application incorporates the entire contents of the foregoing application(s) herein by reference.

The subject matter of this application may have common inventorship with and/or may be related to the subject matter of the following:
U.S. application Ser. No. 15/425,982, titled “Robotically Augmented Catheter Manipulation Handle,” filed by Ryan J. Douglas, et al., on Feb. 6, 2017, and issued as U.S. patent Ser. No. 10/675,442 on Jun. 9, 2020;
U.S. application Ser. No. 16/861,633, titled “Robotically Augmented Catheter Manipulation Handle,” filed by Ryan J. Douglas, et al., on Apr. 29, 2020;
U.S. Provisional Application Ser. No. 62/292,699, titled “ROBOTICALLY ASSISTED STEERABLE CATHETER,” filed by Ryan Douglas, et al., on Feb. 8, 2016; and
U.S. Application Ser. No. 63/111,408, titled “STEERABLE TIP CATHETER WITH AUTOMATIC TENSION APPARATUS,” filed by John Pocmich, et al., on Nov. 9, 2020.

This application incorporates the entire contents of the foregoing document(s) herein by reference.

### TECHNICAL FIELD

Various embodiments relate generally to structures formed in vivo.

### BACKGROUND

Living creatures such as humans and animals are made up of living tissues. The tissues make up various vital organs and systems. Vital systems may include, by way of example and not limitation, cardiovascular, digestive, respiratory, nervous, musculoskeletal, and skin.

Defects may exist in a living tissue. For example, a tissue may develop a defect, such as by injury and/or disease. A creature may be born with a (congenital) defect. Such defects may include an aperture in a tissue. Apertures may, for example, provide an open passageway in a tissue, between two tissues, or some combination thereof.

### SUMMARY

Apparatus and associated methods relate to closure of a stoma with a structure continuously formed in vivo. In an illustrative example, a stoma closure tool (SCT) may include a drive module, a phase transition inducement module (PTIM), and a conduit that defines a lumen. A distal end of the conduit may, for example, be inserted through a first tissue and into a second tissue that together at least partially define a stoma. A flow rate of a fluid biomaterial through the lumen and discharged at the distal end of the conduit may, for example, be controlled by the drive module. A fluid to solid phase transition in the biomaterial may, for example, be controllably induced by the PTIM. Various embodiments may, for example, advantageously form a continuous structure extending directly across the stoma between a proximal anchor in the first tissue and a distal anchor in the second tissue.

Various embodiments may achieve one or more advantages. For example, some embodiments may advantageously close one or more stomata by a resulting unitary stoma closure structure. A unitary stoma closure structure (USCS) may, for example, be advantageously formed in vivo, customized to the particular patient and/or stoma. A unitary stoma closure structure may be advantageously formed, for example, in a minimally invasive procedure. In various embodiments closure of a stoma by a USCS(s) may, for example, advantageously initiate and/or support regenerative remodeling.

The details of various embodiments are set forth in the accompanying drawings and the description below. Other features and advantages will be apparent from the description and drawings, and from the claims.

### BRIEF DESCRIPTION OF THE DRAWINGS

FIG. 1 depicts an exemplary stoma closure tool (SCT) employed in an illustrative use-case scenario.

FIG. 2 depicts an exemplary block diagram of an exemplary stoma closure tool.

FIG. 3 depicts an exemplary block diagram of an exemplary robotic stoma closure tool.

FIG. 4A depicts an exemplary robotic stoma closure tool with an exemplary handheld drive module.

FIG. 4B depicts an exemplary robotic stoma closure tool with an exemplary external drive module.

FIG. 4C depicts an exemplary robotic stoma closure tool with an exemplary robotic drive module.

FIG. 5 depicts an exemplary stoma closure method in an exemplary stoma.

FIG. 6 depicts a flowchart of an exemplary stoma closure method.

FIG. 7 depicts a flowchart of an exemplary method of forming a unitary stoma closure structure.

FIG. 8 depicts exemplary unitary stoma closure structures deployed in exemplary stomas.

FIG. 9 depicts exemplary conduit tips of exemplary stoma closure tools.

FIG. 10 depicts exemplary unitary stoma closure structure segments.

FIG. 11 depicts exemplary closure tensioning modules of exemplary stoma closure tools.

Like reference symbols in the various drawings indicate like elements.

### DETAILED DESCRIPTION OF ILLUSTRATIVE EMBODIMENTS

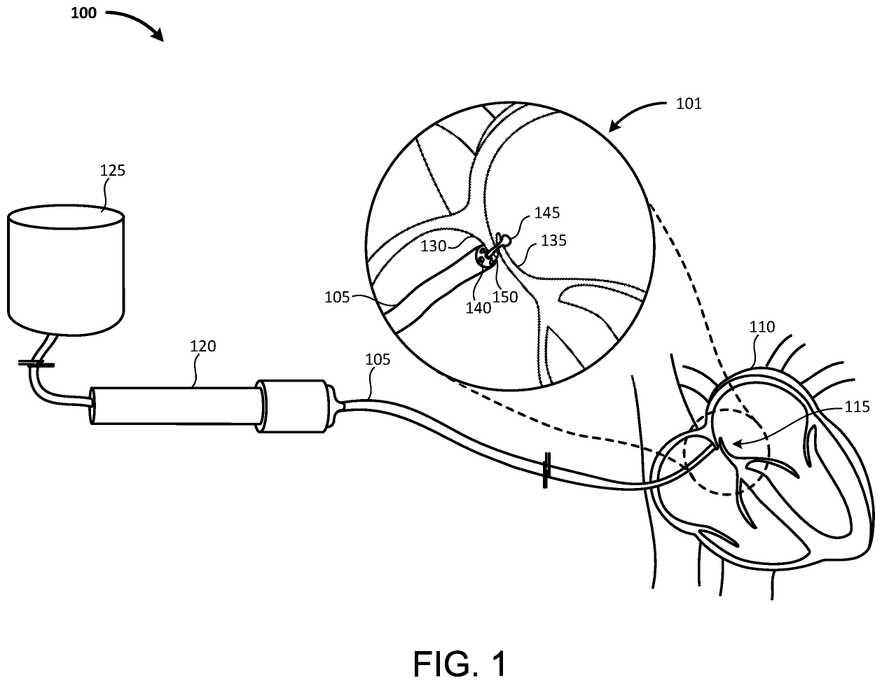

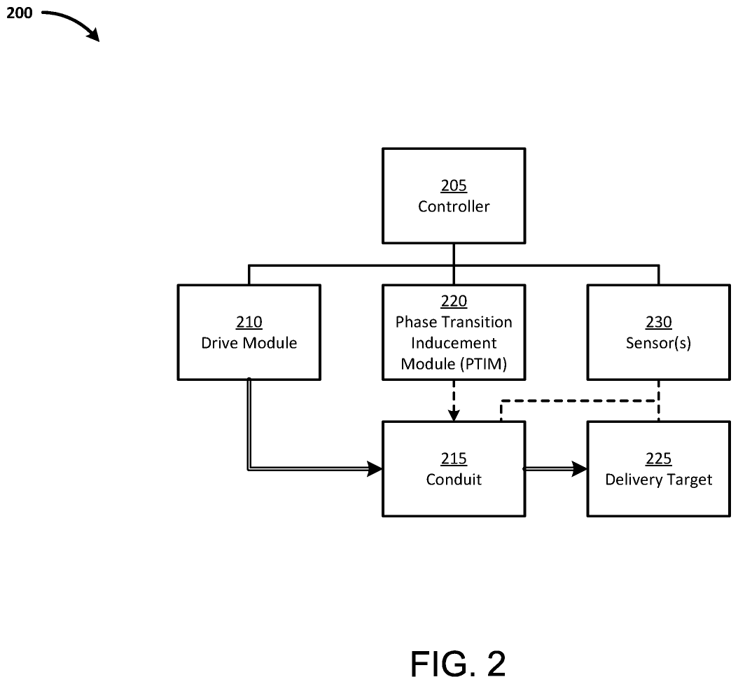

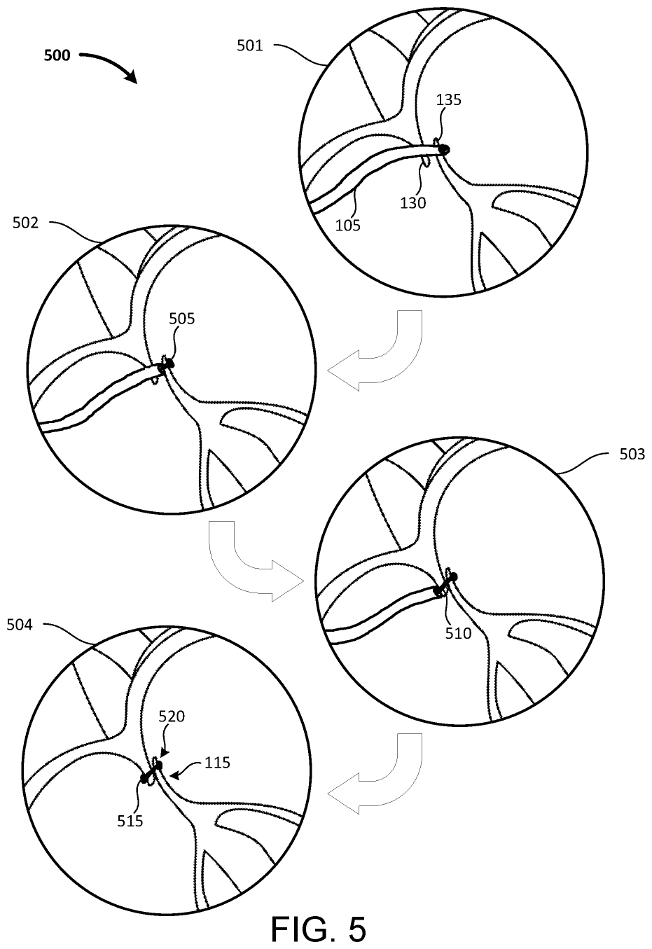

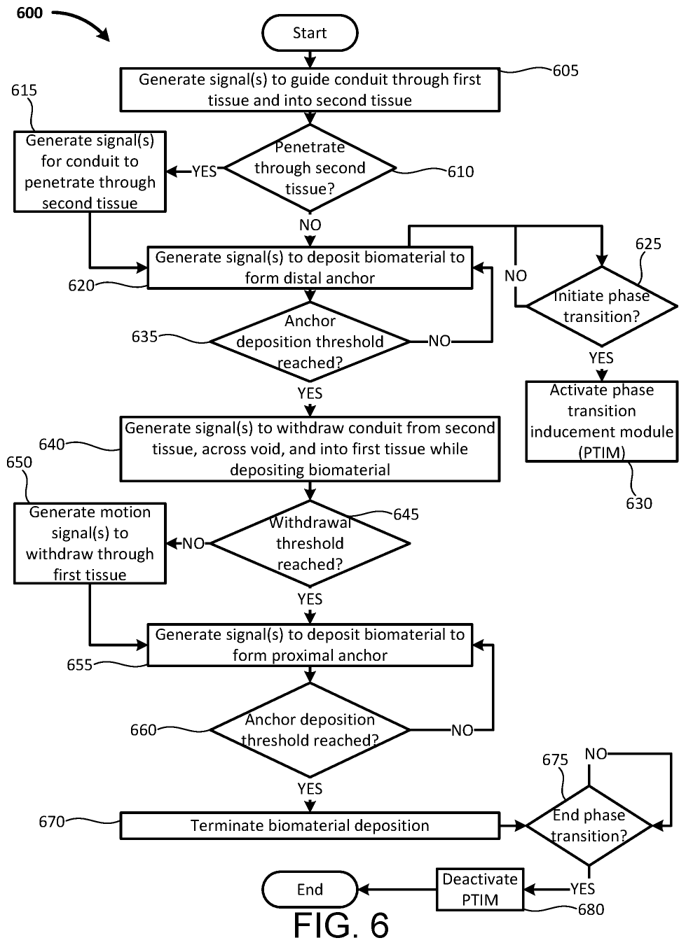

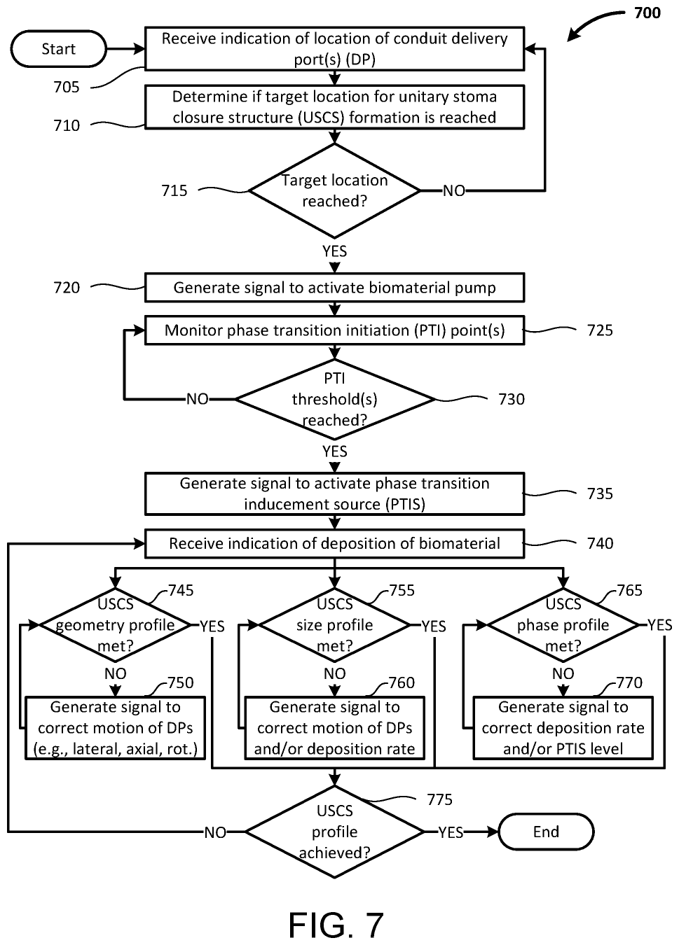

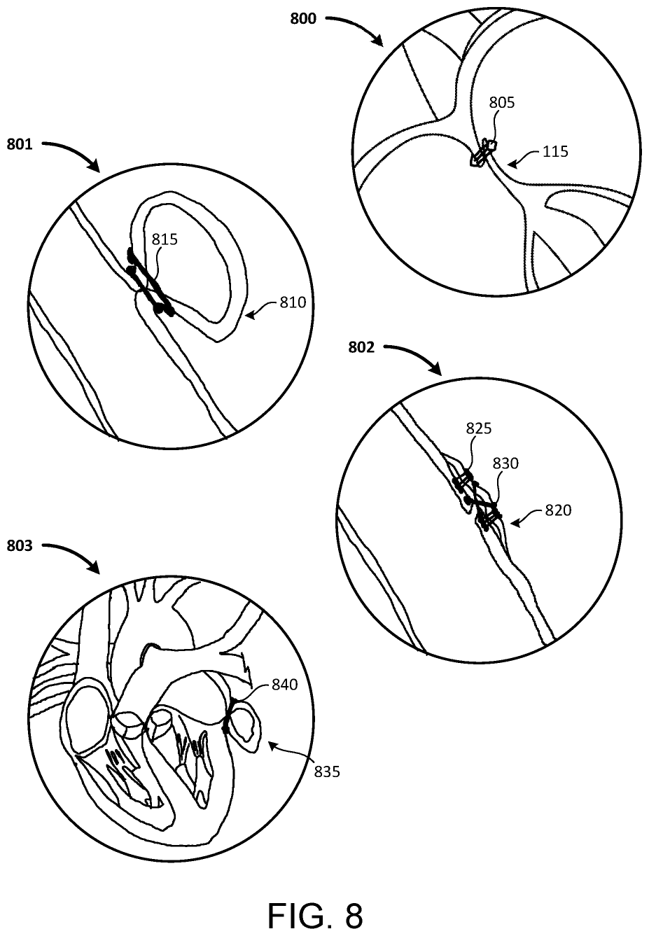

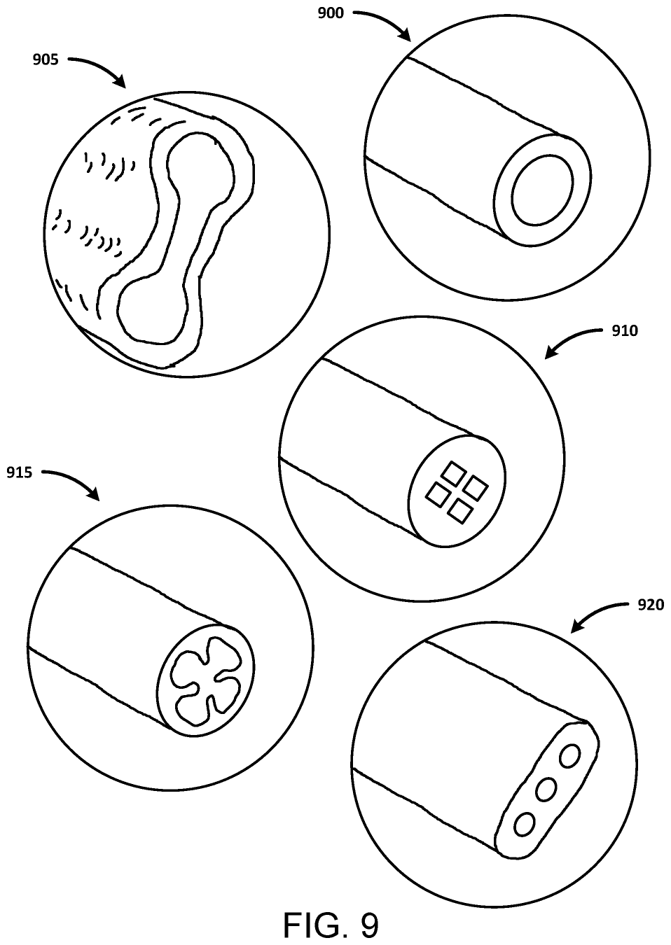

To aid understanding, this document is organized as follows. First, to help introduce discussion of various embodiments, an exemplary stoma closure system is introduced with reference to FIGS. 1-2. Second, that introduction leads into a description with reference to FIGS. 2-4C of some exemplary embodiments of robotic stoma closure systems. Third, with reference to FIGS. 5-6, exemplary methods of stoma closure are described. Fourth, with reference to FIG. 7, the discussion turns to exemplary embodiments of stoma closures as applied to exemplary stomata. Fifth, and with reference to FIGS. 8-9, exemplary stoma closure tool tips and corresponding unitary closures are disclosed. Sixth, and with reference to FIG. to, the disclosure turns to exemplary tensioning modules. Finally, the document discusses further embodiments, exemplary applications and aspects relating to in vivo stoma closure.

FIG. 1 depicts an exemplary stoma closure tool (SCT) employed in an illustrative use-case scenario. In the exemplary depicted scenario 100, a conduit 105 (e.g., a steerable catheter) is inserted (e.g., surgically) into a heart 110 of a patient. A distal end of the conduit 105 engages a stoma 115. In the depicted example, the stoma 115 is a patent foramen ovale.

A proximal end of the conduit 105 is (fluidly) coupled to a catheter handle 120 (e.g., steerable). The proximal end of the conduit 105 may, for example, be releasably or permanently coupled to the catheter handle 120. The conduit 105 is fluidly coupled to a reservoir 125 of biomaterial. The reservoir 125 may include one or more compartments of biomaterial components. A drive module (not shown) may be configured to induce flow of the biomaterial from the reservoir 125, through the conduit 105, and out the distal end of the conduit 105. In various embodiments the drive module may, by way of example and not limitation, be provided in the reservoir 125, the catheter handle 120, coupled directly to the conduit 105, or some combination thereof.

In a closeup view 101 of the stoma 115, a penetration has been made through a first tissue 130 defining the stoma 115 and through a second tissue 135 defining the stoma. Biomaterial has been extruded from a lumen of the conduit 105 upon penetrating the first tissue 130 and the second tissue 135 across the stoma 115. In some embodiments the first tissue 130 and the second tissue 135 may, for example, be two separate tissues. In such embodiments the first tissue 130 and the second tissue 135 may, for example, be sections of the same larger tissue and/or organ.

A phase transition inducement module 140 operates on the biomaterial such that the biomaterial undergoes a phase transition from fluid (e.g., liquid) solid. As depicted, the phase transition inducement module 140 includes multiple light generation modules (e.g., LEDs). The biomaterial may, for example, be photopolymeric. Accordingly, (selective) activation of the phase transition inducement module 140 may induce polymerization of the biomaterial.

Accordingly, a distal anchor 145 (e.g., extruded as (partially) fluid and phase transitioned to solid) is formed in vivo distally of the second tissue 135 and extruded to form a continuous, unitary structure through the second tissue 135, across the stoma 115, and through the first tissue 130. Subsequently, for example, a proximal anchor may be continuously formed connected by a bridge 150 of biomaterial formed continuously between the distal anchor 145 and the proximal anchor. Accordingly, the stoma 115 may be advantageously closed by the resulting unitary stoma closure (structure). The unitary stoma closure may be advantageously formed in vivo, customized to the particular patient and/or stoma. A unitary stoma closure structure may be advantageously formed, for example, in a minimally invasive procedure. The process may be repeated, by way of example and not limitation, to form multiple unitary stoma closures (structures) across the stoma 115 and/or other stomata.

FIG. 2 depicts an exemplary block diagram of an exemplary stoma closure tool. In an exemplary stoma closure system 200, a stoma closure tool is provided with a controller 205. The controller 205 is operably coupled (e.g., electrically and/or mechanically connected) to a drive module 210. The drive module 210 is fluidly coupled (depicted by the double-line connection) to a conduit 215.

The controller 205 is further operably (e.g., electrically and/or mechanically) coupled to a phase transition inducement module 220. The phase transition inducement module 220 is configured to operate on (depicted by the dashed line connection) the contents of the conduit 215. For example, the phase transition inducement module 220 may be disposed and operated to induce a phase transition in material in the conduit 215, exiting the conduit 215, or some combination thereof. Accordingly, a (bio)material may be fluidly delivered through the conduit to a delivery target 225 and induced to transition into a solid substantially upon delivery. The delivery target may, for example, be tissue about and/or defining a stoma.

In the depicted exemplary stoma closure system 200, the controller 205 is further operably (e.g., electrically and/or mechanically) coupled to at least one sensor 230. At least one sensor 230 is configured to monitor the conduit 215. At least one at least one sensor 230 is configured to monitor the delivery target 225. The sensor 230 may, by way of example and not limitation, include a position sensor (e.g., monitoring a position of the conduit 215), motion sensor (e.g., monitoring motion of the conduit 215 and/or the material), proximity sensor, optical sensor (e.g., providing feedback to an operator), force sensor (e.g., monitoring force at a tip of the conduit 215, monitoring fill level of a tissue with dispensed biomaterial), temperature sensor (e.g., monitoring a phase transition status of the material), or some combination thereof.

Accordingly, feedback may be provided to the controller 205. The controller 205 may, for example, generate command(s) in response to feedback from the at least one sensor 230. The controller 205 may, by way of example and not limitation, generate command signals for the drive module 210 (e.g., operate on, operate off, increase and/or decrease dispensing volume, speed, and/or force), the phase transition inducement module 220 (e.g., operate on, operate off, increase actuation and/or dispensing, decrease actuation and/or dispensing), the conduit 215, the sensor(s) 230, or some combination thereof. In some embodiments, the controller 205 may, for example, cause feedback (e.g., visual, audio, tactile) to be provided to an operator (e.g., a physician) and/or receive inputs (e.g., command signals) therefrom. A human machine interface (not shown) may, for example, transduce inputs from the operator into mechanical, pneumatic, hydraulic, and/or electrical signals (e.g., via the controller 205).

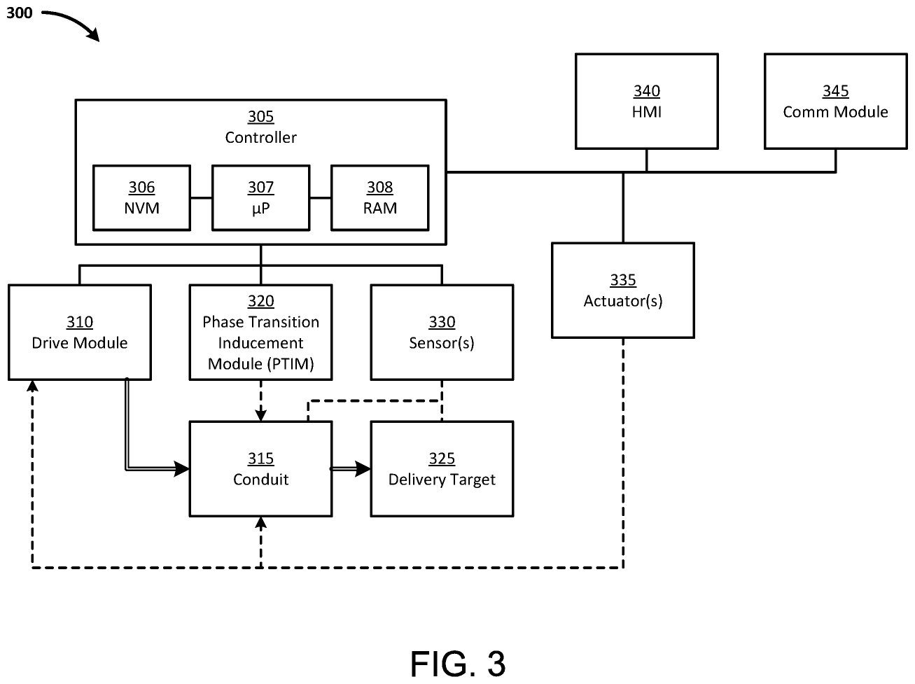

FIG. 3 depicts an exemplary block diagram of an exemplary robotic stoma closure tool. An exemplary robotic stoma closure system 300 includes a controller 305. The controller 305 is provided with a non-volatile memory module 306 (“NVM”), a microprocessor 307 (“μP”), and a random-access memory module 308 (“RAM”). The non-volatile memory module 306 is electrically coupled to the microprocessor 307. The microprocessor 307 is electrically coupled to the random-access memory module 308. The non-volatile memory module 306 may, for example, store one or more programs of instruction which, when executed by the microprocessor 307, perform operations to close a stoma by at least one unitary stoma closure (structure).

The controller 305 is operably (e.g., electrically and/or mechanically) coupled to a drive module 310. The drive module 310 is fluidly coupled to a conduit 315. The conduit 315 may, for example, define at least one lumen configured to transport (bio)material impelled by the drive module 310.

The controller 305 is further operably (e.g., electrically and/or mechanically) coupled to a phase transition inducement module 320. The phase transition inducement module 320 is configured to operate on the material conveyed through the conduit 315 (e.g., in the conduit 315 and/or upon exit from the conduit 315). Accordingly, material may be delivered via the conduit 315 to a delivery target 325 and be transitioned into a solid structure deposited in a desired location and/or configuration.

The controller 305 is further operably (e.g., electrically and/or mechanically) coupled to a sensor 330 (e.g., one or more sensors). In the depicted exemplary robotic stoma closure system 300, at least one of the sensor 330 is configured to monitor the conduit 315 and at least one of the sensor 330 is configured to monitor the delivery target 325. Accordingly, the controller 305 may, by way of example and not limitation, advantageously receive feedback via the sensor 330 regarding the conduit 315, the material, and/or the delivery target 325.

The controller 305 is further operably (e.g., electrically and/or mechanically) coupled to an actuator 335 (e.g., one or more actuators). The actuator 335 is configured to act upon at least one of the drive module 310 and/or the conduit 315. For example, the actuator 335 may be configured to act upon and/or be a component of the drive module 310. Accordingly, at least one actuator 335 may induce flow of the material through the conduit 315 (e.g., from a reservoir). The actuator 335 may, for example, be configured to act upon the conduit 315. For example, the drive module 310 and/or the actuator 335 may be a peristaltic pump operating on the conduit 315 to induce flow of the biomaterial. The actuator 335 may, for example, induce motion of the conduit 315 (e.g., steering the conduit 315, advancing and/or withdrawing the conduit 315, positioning the conduit 315). In various embodiments an actuator 335 may, by way of example and not limitation, operate other modules. Other modules may include, by way of example and not limitation, the phase transition inducement module 320 (e.g., dispensing a phase transition inducing component, operating a light shutter and/or electrical contact, operating a thermal element), an end module on the conduit 315 (e.g., a piercing tool(s), a cutting tool(s), a suture tool(s), a sensor such as an optical sensor), or some combination thereof.

The controller 305 is further operably coupled (e.g., electrically and/or mechanically) to a human-machine interface 340 (“HMI”). The human-machine interface 340 may, by way of example and not limitation, include a button, switch, knob, lever, slider, touch screen, display, mobile device (e.g., smartphone, laptop, tablet), or some combination thereof. For example, a human-machine interface 340 may include a remote-control interface. The human-machine interface 340 may, for example, be provided by an app running on a computing device (such as a mobile device and/or computing device).

The human-machine interface 340 may, for example, be configured to transduce input(s) from an operator into command and/or feedback signals to the controller 305. The human-machine interface 340 may, for example, be configured to generate feedback signals (e.g., visual, audio, tactile) for an operator in response to command signals from the controller 305. For example, in some embodiments the human-machine interface 340 may provide a visual display of formation of a unitary stoma closure (e.g., from an optical sensor such as a camera at a distal end of the conduit 315). In some embodiments the human-machine interface 340 may, for example, receive commands from the operator (e.g., related to steering the conduit 315, advancing and/or withdrawing the conduit 315, operating the drive module 310, operating the phase transition inducement module 320).

The controller 305 is further operably coupled (e.g., electrically and/or mechanically) to a communication module 345. The communication module 345 may, for example, be configured to communicate (e.g., receive and/or transmit signals) between the controller 305 and one or more external devices. The communication module 345 may, for example, communicate with an (external) HMI, with one or more portable devices (e.g., smartphones, tablets, remote control units), servers, computing devices, other medical tools (e.g., robotic catheter system), or some combination thereof. In various embodiments the communication module 345 may, for example, be provided with wired and/or wireless (e.g., Bluetooth, Wi-Fi) communication ports. Ports may, for example, be permanently connected. Ports may, for example, be pluggably connected (e.g., USB, HDMI, RJ45).

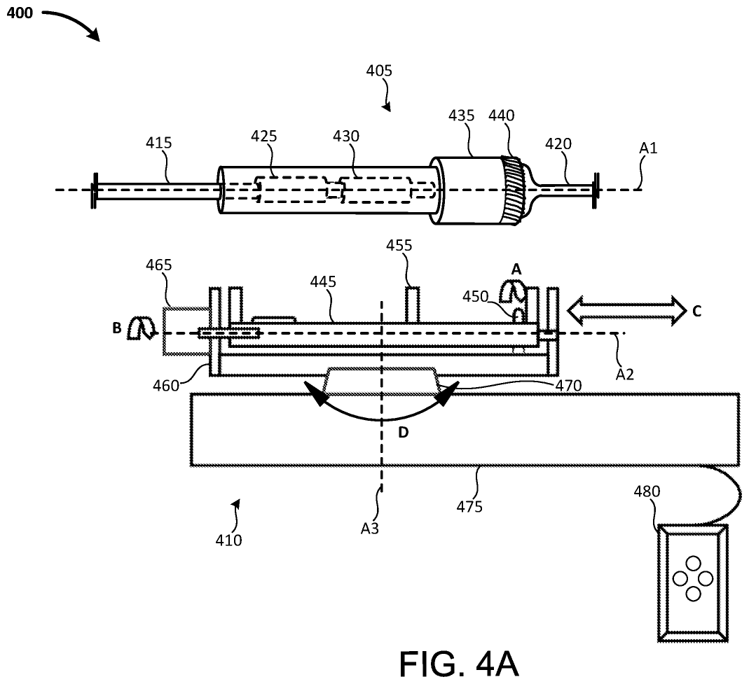

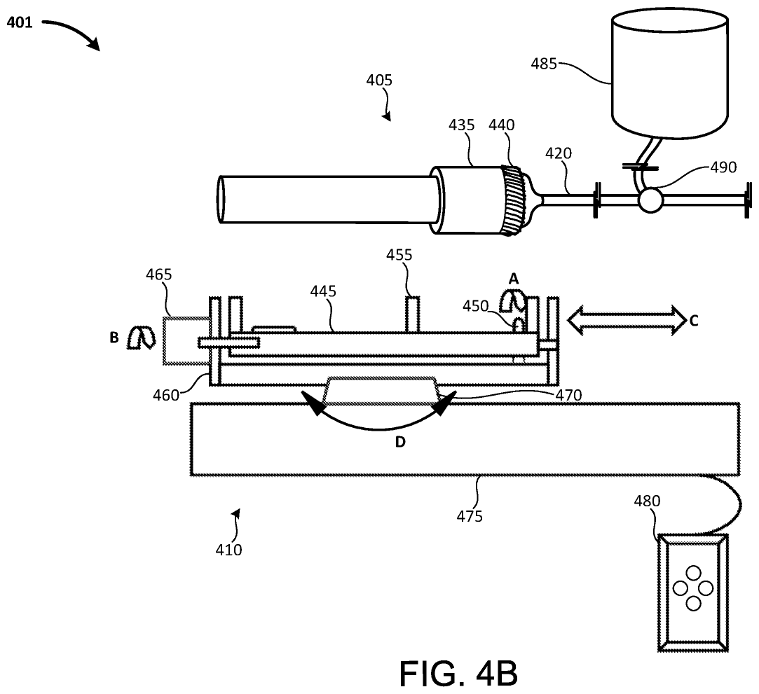

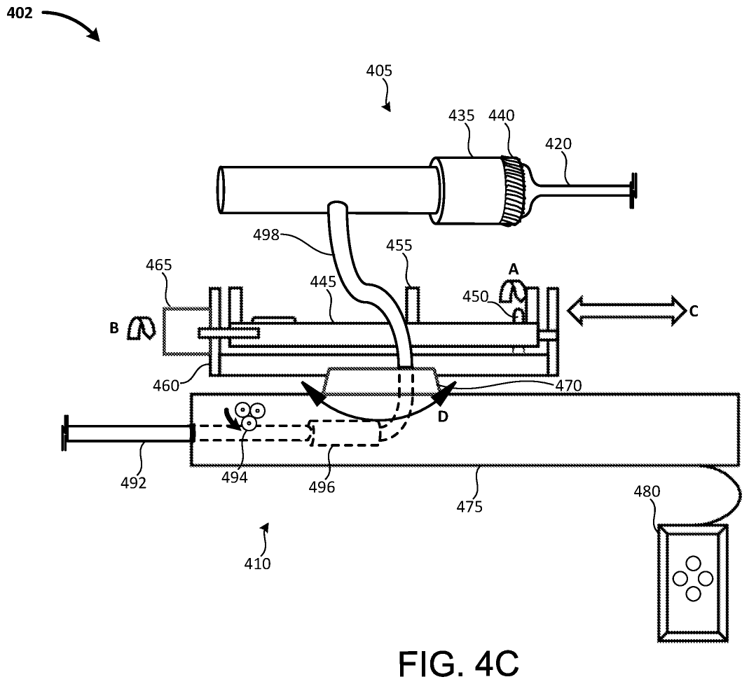

FIG. 4A depicts an exemplary robotic stoma closure tool with an exemplary handheld drive module. In the depicted exemplary stoma closure system 400, a body 405 of a handheld stoma closure tool is configured to be removably mounted to a robotic conduit steering module 410. The robotic conduit steering module 410 may, for example, include a controller (e.g., such as controller 305), one or more actuators (e.g., actuator 335), one or more communication modules (e.g., communication module 345), or some combination thereof.

The body 405 is provided with an inlet conduit 415. The inlet conduit 415 may, for example, be fluidly coupled to a source of biomaterial. Inlet conduit 415 is in fluid communication with an outlet conduit (e.g., for delivery of the biomaterial to a target location such as delivery target 225 and/or delivery target 325).

Disposed within the body 405, and operably coupled to the inlet conduit 415, is a drive module 425. The drive module 425 may, for example, be fluidly coupled to the inlet conduit 415 to receive fluid therefrom and impel it towards the outlet conduit 420. The drive module 425, for example, operate mechanically (e.g., by cyclical pressure) on the inlet conduit 415 to drive biomaterial therethrough. In various embodiments the drive module 425 may include, by way of example and not limitation, an impeller pump, a syringe pump, a peristaltic pump, a diaphragm pump, or some combination thereof.

The drive module 425 may, for example, be operably (e.g., electrically) coupled to at least one controller (e.g., controller 305). The controller may be, by way of example and not limitation, disposed in the body 405, robotic conduit steering module 410, or some combination thereof. The drive module 425 may include, for example, at least one sensor (e.g., sensor 330) and/or actuator (e.g., actuator 335). For example, a sensor(s) may be configured to monitor volume dispensed, force applied during dispensing, volume of biomaterial remaining, flow rate (“Q”), shear stress, pressure, or some combination thereof. An actuator may, for example, be configured to dispense and/or withdraw (e.g., by generating a suction) biomaterial. The actuator may, for example, be operated by the controller at least partially in response to signals received from the sensor.

As an illustrative example, in some embodiments the inlet conduit 415 may be omitted. The body 405 may receive a (replaceable) cartridge of biomaterial. The drive module 425 may operate the cartridge to dispense the biomaterial into the outlet conduit 420. The cartridge may, for example, include at least one syringe. In some embodiments the cartridge may, for example, be integral with the body 405.

The body 405 is further provided with a phase transition inducement module 430. The phase transition inducement module 430 is configured to operate on biomaterial in the inlet conduit 415. The phase transition inducement module 430 may, for example, be fluidly coupled to the inlet conduit 415 and/or the outlet conduit 420. The phase transition inducement module 430 may, for example, be optically and/or thermally coupled to the inlet conduit 415 and/or the outlet conduit 420.

The phase transition inducement module 430 may, for example, be operably (e.g., electrically) coupled to at least one controller (e.g., controller 305). The controller may be, by way of example and not limitation, disposed in the body 405, robotic conduit steering module 410, or some combination thereof. The phase transition inducement module 430 may include, for example, at least one sensor (e.g., sensor 330) and/or actuator (e.g., actuator 335). For example, a sensor(s) may be configured to monitor material properties (e.g., viscosity, density, translucence), flow rate (“Q”), shear stress, pressure, or some combination thereof. At least one actuator may, for example, be configured to dispense a phase transition induction agent, operate a thermal element, operate an optical element, or some combination thereof. The actuator may, for example, be operated by the controller at least partially in response to signals received from the sensor.

In various embodiments the phase transition inducement module 430 may, by way of example and not limitation, include optical elements (e.g., light-emitting elements), thermal elements (e.g., heat emitting and/or heat absorbing elements), chemical dispensing elements (e.g., reservoir, conduit, and/or mixing element to apply phase transition inducing agents into a base biomaterial). Chemical agents may, by way of example and not limitation, include hardeners (e.g., of biocompatible resins), enzymes (e.g., configured to induce coagulation), catalysts, or some combination thereof. In some embodiments the inlet conduit 415 may define multiple lumens. The phase transition inducement module 430 may mix at least two of the lumens together to initiate a phase transition.

In various embodiments, for example, the inlet conduit 415 may define at least two lumens. The phase transition inducement module 430 may mix contents of at least two of the lumens (e.g., actively and/or passively). For example, the phase transition inducement module 430 may include a spiraled mixing chamber configured to induce mixing of contents from the at least two lumens.

In various embodiments the phase transition inducement module 430 may include, by way of example and not limitation, light emitting modules (e.g., LEDs). The light emitting modules may, for example, induce polymerization of a photopolymeric material. The light emitting modules may, for example, be operated continuously. The light emitting modules may, for example, be operated in response to at least one sensor. The light emitting modules may be operated according to manual inputs of an operator.

In various embodiments the phase transition inducement module 430 may include, by way of example and not limitation, thermal modules (e.g., a heater element). The thermal modules may, for example, induce polymerization (e.g., by cross-linking) of a thermoset material. In some embodiments thermal modules may be configured to maintain a (predetermined) minimum temperature threshold of a thermoplastic biomaterial until a predetermined phase transition inducement (PTI) point(s) (e.g., a (predetermined) distance from a delivery port of the outlet conduit 420). The phase transition inducement module 430 may, for example, include (cooling) thermal modules configured to cool the biomaterial to a predetermined maximum temperature threshold to induce solidification (e.g., cross-linking).

The thermal modules may, for example, be operated continuously. The thermal modules may, for example, be operated in response to at least one sensor. The thermal modules may be operated according to manual inputs of an operator.

The body 405 includes a control module 435. The control module 435 is provided with a steering element 440. The control module 435 may, for example, be operably coupled to control an orientation and/or geometry of the outlet conduit 420 (e.g., a steerable catheter). For example, the control module 435 may, in response to rotation relative to the body 405 about a longitudinal axis A1, induce deflection (e.g., mechanically and/or electrically) in the outlet conduit 420.

The robotic conduit steering module 410 is provided with a carriage 445 configured to receive the body 405. An actuation element 450 may be configured to releasably engage the steering element 440. For example, the actuation element 450 may rotate (e.g., driven by an actuator 335 such as an electric motor). The carriage 445 includes a coupling member 455 which may be configured to releasably couple to the body 405 (e.g., behind the control module 435). Accordingly, the body 405 may be releasably axially and rotationally coupled to the carriage 445.

The control module 435 may be rotatable about A1 relative to the body 405 and the carriage 445. The steering element 440 may be held engaged against the actuation element 450. A pattern on the steering element 440 may, for example, be complementary to a pattern (not shown) on the actuation element 450. The actuation element 450 may rotate, as shown by motion “A” (e.g., in response to a command from an operator, such as through the controller 305), thereby inducing rotation about A1 of the control module 435 via the steering element 440 (e.g., having a gear-tooth pattern). Accordingly, the control module 435 may rotate relative to the body 405, thereby inducing a (desired) deflection in the outlet conduit 420. A dispensing port (e.g., a distal end) of the outlet conduit 420 (which may, for example, be at least part of the conduit 315) may thereby be advantageously directed to the delivery target 325.

The carriage 445 may be rotatably coupled to a frame 460. For example, the carriage 445 may be configured to rotate (motion “B”) relative to the frame 460 about a longitudinal axis A2 of the carriage 445. A carriage actuator 465 is operably coupled to the carriage 445 such that operation of the carriage actuator 465 may induce rotation of the frame 460 about the longitudinal axis of the carriage 445. The carriage actuator 465 may, for example, be an actuator 335. The carriage 445 may, for example, be operated by the controller 305 (e.g., in response to commands of an operator). Accordingly, (controlled) rotation of the body 405 may be advantageously induced about A2 when the body 405 is releasably coupled to the carriage 445.

The frame 460 is coupled to an upper base 470. For example, the frame 460 may be slidably and/or rotatably coupled to the upper base 470. An actuator (not shown, such as, for example, an actuator 335) may be configured to advance and/or retract the frame 460 along A2 relative to the upper base 470 (motion “C”), to rotate the frame 460 about an axis A3 relative to the upper base 470 (motion “D”), or some combination thereof.

The upper base 470 is coupled to a lower base 475. The upper base 470 may, for example, be rotatably and/or slidably coupled to the lower base 475. An actuator (not shown, such as, for example, an actuator 335) may be configured to advance and/or retract the upper base 470 along A2 relative to the lower base 475 (motion “C”), to rotate the lower base 475 about A3 relative to the lower base 475 (motion “D”), or some combination thereof.

In some embodiments, for example, the frame 460 may rotate about A3 relative to the upper base 470 and the upper base 470 may translate along A2 relative to the lower base 475. In some embodiments, for example, the frame 460 may translate along A2 relative to the upper base 470 and the upper base 470 may rotate about A3 relative to the lower base 475.

The lower base 475 is operably coupled to a human machine interface 480. As depicted, the human machine interface 480 is in wired electrical communication with the lower base 475. For example, the human machine interface 480 may be at least part of a human-machine interface 340. The human machine interface 480 may, for example, be coupled to the controller 305. The human machine interface 480 may, for example, be coupled to the controller 305 via the communication module 345.

In such embodiments, the human machine interface 480 may, by way of example and not limitation, be wirelessly coupled to the controller 305 (e.g., in the robotic conduit steering module 410). The human machine interface 480 may, for example, be a dedicated HMI. The human machine interface 480 may, for example, be a multipurpose HMI (e.g., a mobile computing device). In some embodiments, the human machine interface 480 may, for example, include a visual feedback (e.g., a display screen), tactile (e.g., haptic) feedback mechanism(s), or some combination thereof. The human machine interface 480 may, for example, transduce (mechanical) inputs from a user into signals provided to the controller 305. The controller 305 may, for example, generate signals to operate the various actuators (e.g., actuator 335, carriage actuator 465, actuator of the actuation element 450, actuator of the frame 460, actuator of the upper base 470, actuator of the drive module 425, actuator of the phase transition inducement module 430). Accordingly, an operator (e.g., a physician) may advantageously operate the body 405 via the human machine interface 480.

Accordingly, various embodiments may advantageously provide multiple (e.g., 2, 3) degrees of freedom of the body 405 (e.g., motion B, C, and D). Various embodiments may advantageously operate the outlet conduit 420 along at least one additional degree of freedom (e.g., 2, 3 degrees of freedom). Accordingly, (robotic) placement of a unitary closure structure at a (precise) desired delivery target (e.g., delivery target 325) may be advantageously achieved.

FIG. 4B depicts an exemplary robotic stoma closure tool with an exemplary external drive module. In the depicted exemplary robotic stoma closure system 401, the body 405 is provided with the outlet conduit 420. A reservoir 485 is fluidly coupled to the outlet conduit 420 by a coupling module 490. The reservoir 485 may, for example, contain (bio)material, a phase transition inducing agent, or some combination thereof.

In various embodiments the reservoir 485 and/or the coupling module 490 may, by way of example and not limitation, include a valve, an actuator, a mixing element, a PTIM, a drive module, or some combination thereof. For example, in an illustrative example, the reservoir 485 may be provided with a drive module (e.g., the drive module 310). The drive module may, for example, induce flow of material from the reservoir 485 through the coupling module 490, and thence through the outlet conduit 420 and out a region of the outlet conduit 420 distal to the coupling module 490. The drive module may, for example, be operably coupled to the controller 305. The drive module 310 may, for example, be configured as disclosed at least with reference to FIG. 4A. The coupling module 490 may, for example, contain a heating element, mixing element, and/or light emitting element of the PTIM. In some embodiments, the PTIM may, for example, be at least partially disposed on, in, and/or about a region of the outlet conduit 420 distal to the coupling module 490 (e.g., at a distal tip of the outlet conduit 420).

In an illustrative embodiment, a source of first material (not shown) may be provided through a region of the outlet conduit 420 proximal to the coupling module 490 (e.g., through the body 405, as disclosed at least with reference to FIG. 4A). A second material may be dispensed from the reservoir 485. The coupling module 490 may, for example, include a valve (e.g., electrically operated by the controller 305 in response to signals from an operator and/or at least one sensor 330). The coupling module 490 may, for example, include mixing features (e.g., protrusions in a chamber into which a lumen of the outlet conduit 420 and a lumen of the conduit from the reservoir 485 both open) configured to mix the first material and the second material. Accordingly, the coupling module 490 may include at least part of the PTIM.

FIG. 4C depicts an exemplary robotic stoma closure tool with an exemplary robotic drive module. In the depicted exemplary robotic stoma closure system 402, the robotic conduit steering module 410 is provided with an inlet conduit 492 configured to pass through the lower base 475. The inlet conduit 492 may, for example, be fluidly coupled to at least one reservoir of (bio)material, phase transition inducing agent, or some combination thereof.

A drive module 494 is provided in the lower base 475. In the depicted exemplary embodiment, the drive module 494 includes a rotating drive member having three wheels. The drive module 494 may rotate about a central axis of the drive module 494, thereby cyclically deforming the inlet conduit 492 (which may, for example, include at least a segment of flexible tubing). Accordingly, material may be induced to flow through the conduit (e.g., forwards, or backwards, depending on the direction of rotation of the drive module 494).

As depicted, the lower base 475 further includes a PTIM 496. The PTIM 496 may, for example, include a mixing module, thermal module, light emitting module, or some combination thereof. The PTIM 496 may, for example, be fluidly coupled in line with the inlet conduit 492. The PTIM 496 may, for example, be disposed and configured to operate through and/or in the inlet conduit 492. For example, the PTIM 496 may include light-emitting elements configured to shine light through a (semi-)transparent wall (portion) of the inlet conduit 492.

The inlet conduit 492 is coupled to the body 405 via an intermediate conduit 498. The intermediate conduit 498 may, for example, be operably fluidly coupled to the outlet conduit 420 through the body 405. Accordingly, material may be advantageously delivered from the inlet conduit 492 through the intermediate conduit 498 and out the outlet conduit 420.

An effective length of conduit from the PTIM 496 (including the intermediate conduit 498) to a delivery port(s) (e.g., a distal end) of the outlet conduit 420 may, by way of example and not limitation, be configured to correspond with a time of phase transition of the material to a desired solidity level at point of delivery (e.g., semi-solid), and/or vice versa.

FIG. 5 depicts an exemplary stoma closure method in an exemplary stoma. In the depicted method 500, the stoma is defined by the first tissue 130 and the second tissue 135 (e.g., as disclosed at least with reference to FIG. 1). The stoma may, for example, be a patent foramen ovale in a human (pediatric) heart. In a first depicted step 501, the conduit 105 is advanced to the stoma and through the first tissue 130 and the second tissue 135. The conduit 105 may, for example, be a catheter having a distal tip configured to pierce tissue and/or an aperture (e.g., a slit) may be formed in the tissue by a separate tool for passage of the distal tip of the conduit 105. A drive module induces flow of biomaterial out of the conduit 105. A PTIM (e.g., the phase transition inducement module 140 disclosed at least with reference to FIG. 1) induces a phase transition from fluid to (at least partially) solid of the biomaterial. For example, the biomaterial may be at least sufficiently solid to remain in place as it exits the conduit 105.

In a second depicted step 502, a distal anchor 505 has been formed. The distal anchor 505 may, for example, be formed by a dwell period of the conduit 105 during dispensing of the biomaterial, by motion (e.g., a circular motion, small advancement and/or retraction motion) of the distal tip of the conduit 105, or some combination thereof. The distal tip of the conduit 105 has been withdrawn from the second tissue 135 while biomaterial continues to be dispensed. The rate of withdrawal and/or a rate of dispensing may, for example, be controlled, such that the biomaterial dispensed continuously forms a continuous, unitary structure with the distal anchor 505.

In a third depicted step 503, the distal tip of the conduit 105 has been withdrawn through the first tissue 130 while continuing to dispense biomaterial. Accordingly, a bridge 510 has been formed which is a continuous, unitary structure with the distal anchor 505.

In a fourth depicted step 504, deposition of the biomaterial has been terminated and the conduit 105 has been withdrawn after formation of a proximal anchor 515. The proximal anchor 515 is a continuous unitary structure with the bridge 510 and the distal anchor 505. For example, the proximal anchor 515 may be formed after and/or during withdrawal through the first tissue 130 by a dwell period of the conduit 105 during dispensing of the biomaterial, by motion (e.g., a circular motion, small advancement and/or retraction motion) of the distal tip of the conduit 105, or some combination thereof.

Accordingly, a continuous, unitary stoma closure structure 520 (USCS) has been formed joining the first tissue 130 and the second tissue 135 together. As depicted, the unitary stoma closure structure 520 thereby advantageously closes the stoma 115.

FIG. 6 depicts a flowchart of an exemplary stoma closure method. A depicted method 600 may, for example, be performed by a processor (e.g., microprocessor 307) executing a program of instructions (e.g., stored on non-volatile memory module 306). Various steps of the depicted method 600 are disclosed at least with reference to FIG. 5 with respect to an exemplary embodiment.

The depicted method 600 begins with a step 605 include generation of one or more signals to cause a conduit to penetrate through a first tissue (e.g., first tissue 130) and into a second tissue (e.g., second tissue 135). The signals may, for example, be motion signals. The motion signals may, for example, be configured as (electrical) command signals to actuators of the robotic conduit steering module 410, the body 405, or some combination thereof. The (motion) signals may be generated, by way of example and not limitation, according to operator inputs (e.g., via the human-machine interface 340 and/or human machine interface 480), according to a predetermined motion trajectory and/or target, or some combination thereof. For example, a predetermined motion trajectory may be defined (e.g., by an operator) prior to initiation of the depicted method 600. The predetermined motion trajectory and/or target may be stored (e.g., on the non-volatile memory module 306) as one or more files (e.g., CNC file such as containing GCode, file containing a sequence of predetermined point(s), file contained a sequence of predetermined motion vectors). The predetermined motion trajectory and/or target may be defined as a function of imaging. In some embodiments the (motion) signal(s) may be generated, for example, dynamically in response to imaging and/or input from other sensors (e.g., one or more sensor 330).

In decision point 610, if the procedure includes penetrating through the second tissue, then a step 615 includes generating one or more (motion) signals configured to cause the conduit to penetrate through the second tissue. In various embodiments, the procedure may, for example, cause the conduit to penetrate entirely through the second tissue in order to position at least one distal anchor against an (outer) surface of the second tissue.

In some embodiments, the procedure may prescribe that a distal anchor should be embedded within the second tissue. The decision point 610 may, therefore, determine that the second tissue should not be penetrated. Accordingly, in a step 620, one or more signals are generated to initiate deposition of biomaterial to form the distal anchor. The signal(s) may, for example, cause operation of a drive module (e.g., drive module 310). The drive module may, for example, be configured as disclosed at least with reference to FIG. 4A.

At a decision point 625, it is determined whether phase transition is to be initiated. If yes, then a signal(s) is generated in a step 630 to activate a PTIM (e.g., phase transition inducement module 320). The PTIM may, for example, be configured as disclosed at least with reference to FIGS. 4A-4C. If phase transition is not to be initiated, the decision point 625 is revisited until phase transition is to be initiated. The point at which phase transition may be initiated may, by way of example and not limitation, be determined according to a predetermined phase transition initiation (PTI) point(s). The PTI point(s) may, by way of example and not limitation, correspond to a phase transition duration (e.g., time required from initiation of phase transition to achieving a desired state of the biomaterial at a point of deposition), an effective length and/or time of travel between the PTI point and deposition, a level of solidity that is required of the material at deposition (e.g., according to a force to be applied, a type of tissue), or some combination thereof. In some embodiments, a PTI point(s) may be dynamically determined such as, for example, according to feedback from a sensor(s) regarding biomaterial characteristics at one or more points (e.g., at a dispensing port of a conduit).

After step 620, it is determined in a decision point 635 whether an anchor deposition threshold(s) has been reached. A first anchor deposition threshold may, by way of example and not limitation, be predetermined and/or dynamic. For example, an anchor deposition threshold may be defined by a (predetermined) UCSS profile. The UCSS profile may prescribe parameters of a USCSS such as, by way of example and not limitation, geometry (e.g., size, shape, orientation) of an anchor(s) and/or bridge(s) of the UCSS, mechanical properties of the UCSS and/or some portion thereof, motion for formation of a UCSS, material deposition rates, phase transition points, dwell times, withdrawal rates, or some combination thereof.

A UCSS profile may, for example, be predetermined. A UCSS profile may, for example, be dynamically determined and/or dynamically modified (e.g., within a predetermined range of values for a particular parameter). A UCSS profile may, for example, define functions by which a parameter may be (dynamically) determined according to one or more other parameters. A library of (predetermined) UCSS profiles may, for example, be provided for an operator (e.g., physician) to select from. The selected UCSS profile may be loaded into at least one NVM module for execution by at least one processor.

The first anchor deposition threshold may, for example, determine a minimum size (e.g., diameter, volume) of the distal anchor. If the first anchor deposition threshold is not determined to be met (e.g., based on feedback from at least one sensor such as sensor 330) in decision point 635, then the depicted method 600 returns to step 620.

Once the first anchor deposition threshold has been determined to be met in decision point 635, then one or more signals are generated in a step 640 causing the conduit to be withdrawn from the second tissue and into the first tissue while continuing to deposit biomaterial. The signals may, for example, include motion signal(s) (e.g., as disclosed with reference to the step 605). The signals may, for example, include command signal(s) to a drive module and/or PTIM. The signals may, for example, be predetermined. The signals may, for example, be dynamic (e.g., in response to feedback from one or more sensors). The signals may, for example, be dynamically determined to adjust the withdrawal rate, material deposition rate, and/or phase transition inducement intensity and/or timing to achieve one or more (predetermined) parameters for the UCSS (e.g., as determined in a UCSS profile). Accordingly, a bridge may be advantageously formed (across the stoma) continuously and unitarily with the distal anchor.

In a decision point 645, it is determined if a withdrawal threshold has been reached. If the withdrawal threshold has not been reached then, in a step 650, further (motion) signals are generated to cause the conduit to be withdrawn through the first tissue (e.g., to deposit a proximal anchor at least partially outside of a first tissue instead of (fully) embedded in the first tissue). Once the withdrawal threshold has been reached, then signals are generated to deposit biomaterial to perform the proximal anchor in a step 655. The signals may, for example, include command signals to a drive module and/or PTIM (e.g., to increase phase transition inducement intensity, such as light intensity to correspond to an increased deposition volume). The signals may, for example, include motion signals to cause dwell, lateral motion, and/or withdrawal and/or advancement of a conduit to form the proximal anchor. The signals may, for example, be generated at least partially according to the UCSS profile.

In a decision point 660, it is determined if a second anchor deposition threshold is reached. The second anchor deposition threshold may be determined by a UCSS profile. Some embodiments the second anchor deposition threshold may be the same as the first anchor deposition threshold. If the second anchor deposition threshold is not reached, then the depicted method 600 returns to the step 655. Once the second anchor deposition threshold is reached, then biomaterial deposition is terminated in a step 670. Termination may, for example, be caused by generation and/or cessation of signals to the drive module. In a decision point 675, if it is determined phase transition should in, then the PTIM is deactivated (e.g., by generation and/or cessation of command signals thereto) in a step 680. The PTIM may, for example, be deactivated before the biomaterial deposition is terminated. The PTIM may, for example, be deactivated after the biomaterial deposition is terminated (e.g., to induce sufficient curing in later deposited material). Once the PTIM is deactivated in the step 680, then the depicted method 600 ends. The conduit may, for example, be subsequently withdrawn.

FIG. 7 depicts a flowchart of an exemplary method of forming a unitary stoma closure structure. A depicted method 600 may, for example, be performed by a processor (e.g., microprocessor 307) executing a program of instructions (e.g., stored on non-volatile memory module 306). Various steps of the depicted method 700 are disclosed at least with reference to FIG. 5 with respect to an exemplary embodiment. The depicted method 700 may, for example, form a portion of the depicted method 600. Various system (components) and/or methods may, for example, be configured and/or performed as disclosed at least with reference to FIGS. 4A-4C.

The depicted method 700 begins with a step 705 and which an indication of location of at least one conduit delivery port (DP) is received. The indication may, for example, be received as signal(s) from one or more sensors. The sensors may, for example, include a (robotic) camera (e.g., disposed on the conduit and configured to monitor the DP), a (separate) imaging module (e.g., radiography module, fluoroscopy module, magnetic resonance imaging module, computed tomography module, ultrasound module), proximity sensor, force sensor, pressure sensor, position and/or orientation sensor (e.g., accelerometer, gyroscope), or some combination thereof.

In a step 710, it is determined if a target location (e.g., operator determined, (pre)determined by a UCSS profile) for formation of the unitary stoma closure structure (UCSS) has been reached. If the target location has not been reached (decision point 715), that a depicted method 700 returns to the step 705. Once the target location has been reached, then at least one signal is generated in a step 720 to activate a biomaterial pump of a drive module. The signal(s) may be generated, for example, according to the UCSS profile.

At least one phase transition initiate (PTI) point is monitored in a step 725. If a PTI threshold has not yet been reached (decision point 730), then the PTI points continue to be monitored in the step 725. For example, the PTI points may correspond to time (e.g., delay), distance (e.g., when a dispensed biomaterial is sensed as having reached a specific portion of the conduit), material properties (e.g., biomaterial density, hardness, force required to advance the biomaterial through the conduit) or some combination thereof. As an illustrative example, the PTI point(s) may correspond to biomaterial reaching at least one of the DP(s). Once one or more PTI points have been reached, then at least one signal is generated in a step 735 to activate a phase transition inducement source (PTIS). The PTIS may, for example, be light-emitting modules of a PTIM. The light-emitting modules may, for example, be disposed at or about the DP(s).

In a step 740, indication is received of deposition of the biomaterial. For example, indication may be received via the robotic camera(s) configured to (visually) monitor one or more of the DP(s). Image analysis may be performed on the received indication (e.g., image(s), video stream). The image analysis may, for example, be according to (predetermined) algorithms configured to determine at least one parameter of the deposited biomaterial (e.g., size, orientation, geometry, phase state).

In a decision point 745, the received indication is analyzed to determine whether a USCS geometry profile has been met. The geometry profile may, for example, be defined by a USCS profile. If the geometry profile has not been met, then one or more signals are generated in a step 750 to correct motion of the DP(s). For example, the signal(s) may be motion signals configured to induce lateral (e.g., deflection) motion, rotational motion (e.g., about a longitudinal axis of the conduit), and/or axial motion (e.g., withdrawal and/or advancement) of the DP(s). In an illustrative example, the motion signal(s) may, for example, operate the robotic conduit steering module 410 to achieve at least one of motions A, B, C, and/or D.

In a decision point 755, the received indication is analyzed to determine whether a USCS size profile has been met. The size profile may, for example, be defined by a USCS profile. If the size profile has not been met, then one or more signals are generated in a step 760 to correct motion (e.g., axial, rotational, and/or lateral), to correct deposition rate of the biomaterial, or some combination thereof. Deposition rate may, for example, be corrected by one or more commands signals to the biomaterial pump of the drive module.

In a decision point 765, the received indication is analyzed to determine whether a USCS phase profile has been met. The phase profile may, be defined by a USCS profile. If the phase profile (e.g., density, hardness, maximum change in shape over a predetermined period of time as determined by image analysis) has not been met, then one or more signals are generated in a step 770 to correct the deposition rate and/or the PTIS level. The PTIS level may, for example, be corrected by one or more command signals to the PTIS of the PTIM.

In a decision point 775, it is determined whether the USCS profile has been achieved. For example, the decision point 775 is determined at least by whether the USCS geometry profile has been determined to be met in the decision point 745, the USCS size profile has been determined to be met in the decision point 755, and the USCS phase profile has been determined to be met in the decision point 765. If not, then the depicted method 700 returns to the step 740. Otherwise, the depicted method 700 may end.

FIG. 8 depicts exemplary unitary stoma closure structures deployed in exemplary stomas. In various embodiments a stoma may, for example, be an aperture (e.g., opening) in a body of a living creature, or tissue of a living creature. In a first illustrative example 800, a first exemplary stoma 115 (e.g., as disclosed at least with reference to FIGS. 1 and 5) is closed by a first exemplary unitary stoma closure structure 805 (USCS). As depicted, the first exemplary unitary stoma closure structure 805 is provided with three-dimensional rectangular distal and proximal anchor points connected by multiple (e.g., at least 3) filamentous bridges. In some embodiments, such a USCS may advantageously reduce a size of a single hole through a first tissue and/or second tissue. The USCS may, for example, be formed by penetration of the tissues by multiple ports, forming the distal anchor (e.g., by fusion of multiple individual anchors before final solidification), and withdrawing to form the filamentous bridges. Accordingly, pressure on the tissue required to maintain closure may, for example, advantageously be distributed across a greater surface area and/or a greater area of contact may be maintained between the first and second tissues. Closure of the stoma 115 by the first exemplary unitary stoma closure structure 805 may, for example, advantageously initiate and/or support regenerative remodeling of the heart tissue to form a continuous septum between the atria.

In a second illustrative example 801, a stoma 810 is developed in a wall of a vessel (e.g., a blood vessel). As depicted, the stoma 810 is a saccular aneurysm. Two USCSs 815 are formed across the neck of the stoma 810. Accordingly, the stoma 810 is closed, and a desired (e.g., original) geometry of the interior of the vessel is substantially restored. Closure of the stoma 810 by the USCSs 815 may, for example, advantageously initiate and/or support regenerative remodeling and formation of a continuous endothelial layer across the closed neck of the stoma 810.

In a third illustrative example 802, a stoma 820 is developed in a wall of a vessel (e.g., a blood vessel). As depicted, the stoma 820 is a pseudo aneurysm. Two diffuse USCSs 825 are formed, each having three filamentous bridges continuously formed with and connected end anchors. The end anchors may, for example, be formed by deposition and fusion of biomaterial from three lumens prior to final solidification. Accordingly, the wall of the stoma 820 may, for example, be advantageously ‘pinned down,’ thereby inducing adhesion to the vessel wall. Two individual USCSs 830 are formed, each having individual end anchors and a single continuously formed connected bridge. The USCSs 830 are configured to connect the ruptured inner wall of the vessel to the outer wall of the stoma 820, thereby effectively closing the gap in the inner wall of the vessel. Closure of the stoma 820 by the USCSs 830 and the USCSs 825 may, for example, advantageously initiate and/or support regenerative remodeling and formation of a continuous endothelial layer across the closed aperture in the inner vessel wall.

In a fourth illustrative example 803, a stoma 835 exists in a left atrium of a human heart. As depicted, the stoma 835 is a left atrial appendage. A USCS 840 is formed across the neck of the stoma 835. The USCS 840 includes ‘clover’-shaped end anchors connected by a bridge (e.g., having a four-lobed cross-sectional profile). The USCS 840 may, for example, provide closure of the stoma 835 of sufficient size and/or strength to withstand the motion of the myocardium. Closure of the stoma 835 by the USCS 840 may, for example, advantageously initiate and/or support regenerative remodeling of the left atrium.

FIG. 9 depicts exemplary conduit tips of exemplary stoma closure tools. A first exemplary conduit tip 900 is substantially circular, defining a substantially circular lumen cross-section (e.g., as disclosed at least with reference to FIGS. 1 and 5. A second illustrative conduit tip 905 defines a lumen cross-section having a flat section connecting two end lobes. A third illustrative conduit tip 910 defines four lumens, each having a substantially square cross-section. A fourth illustrative conduit tip 915 defines a lumen having a cross-section defined by four connected lobes (e.g., ‘clover-shaped’). A fifth illustrative conduit tip 920 defines three lumens, each having a substantially circular cross-section.

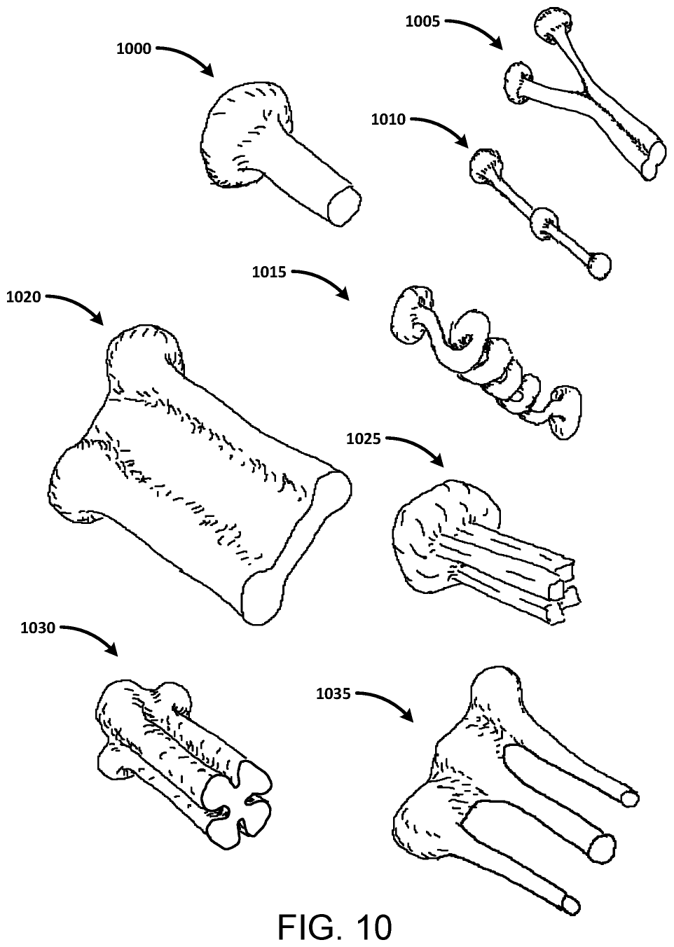

FIG. 10 depicts exemplary unitary stoma closure structure segments. A first illustrative USCS segment 1000 is formed having a ‘mushroom-shaped’ anchor continuously and unitarily formed with a single bridge. The first illustrative USCS segment 1000 may, for example, be formed by the first exemplary conduit tip 900.

A second illustrative USCS segment 1005 is formed by having two individual anchors, each continuously and unitarily formed with a single bridge. Two single bridges are joined along their length at a distance from the anchors. The second illustrative USCS segment 1005 may, for example, be formed with the first exemplary conduit tip 900 by forming a first anchor and bridge followed by a second anchor and bridge, during a phase transition period before the material was (completely) solidified such that the two bridges at least partially fused together (e.g., by cross-linking). The second illustrative USCS segment 1005 may, for example, be formed simultaneously by two conduit tips. For example, a conduit may be provided with individually controllable tips such that the individual tips may be spread apart to form the anchors and then advanced closer together until the bridges join. The second illustrative USCS segment 1005 may, for example, advantageously provide multiple anchor points in and/or against a tissue(s) for a single (effective) bridge.

A third illustrative USCS 1010 is formed by two individual anchors continuously and unitarily formed with a single bridge and having a spacing element continuously and unitarily formed between the two anchors. The third illustrative USCS 1010 may, for example, be formed by the first exemplary conduit tip 900. The spacing element may, for example, advantageously maintain a desired distance between two tissues being joined.

A fourth illustrative USCS 1015 is formed by two individual anchors continuously and unitarily formed with a spiraled bridge. The fourth illustrative USCS 1015 may, for example, be formed by the first exemplary conduit tip 900. The spiraled bridge may, for example, be formed by synchronized deflection and rotation of a conduit tip during insertion and/or withdrawal. In some embodiments the spiraled bridge may, for example, be formed by a ‘corkscrew’ tip which may be inserted by rotation (e.g., synchronized with linear translation along a longitudinal axis) of the tip to ‘screw’ the tip into the tissue. The fourth illustrative USCS 1015 may, for example, advantageously ‘suture’ two tissues together along their length.

A fifth illustrative USCS 1020 is formed with the cross-section having a substantially flat portion connecting two lobular ends. An anchor is formed of a larger version of the cross-section. The fifth illustrative USCS 1020 may, for example, be formed by the second illustrative conduit tip 905. The fifth illustrative USCS 1020 may, for example, advantageously provide a strong, ‘strap-like’ USCS for closure of large and/or (relatively) wide apertures.

A sixth illustrative USCS 1025 is formed with a substantially three dimensionally rectangular anchor unitarily and continuously formed with four bridges having substantially square cross-sections. The sixth illustrative USCS 1025 may, for example, be formed by the third illustrative conduit tip 910. The sixth illustrative USCS 1025 may, for example, advantageously provide relatively large end anchors connected by multiple bridges).

A seventh illustrative USCS 1030 is formed with a three-dimensionally four-lobular anchor unitarily and continuously formed with a bridge having a four-lobed cross-section. The seventh illustrative USCS 1030 may, for example, be formed by the fourth illustrative conduit tip 915. The seventh illustrative USCS 1030 may, for example, provide a robust USCS which may, for example, advantageously resist axial bending in at least two planes.

An eighth illustrative USCS 1035 is formed with an end anchor unitarily and continuously formed with three (filamentous) bridges. The eighth illustrative USCS 1035 may, for example, be formed by the fifth illustrative conduit tip 920. The eighth illustrative USCS 1035 may, for example, provide a relatively thin, wide bearing surface and/or embedment for the anchors, coupled by multiple bridges.

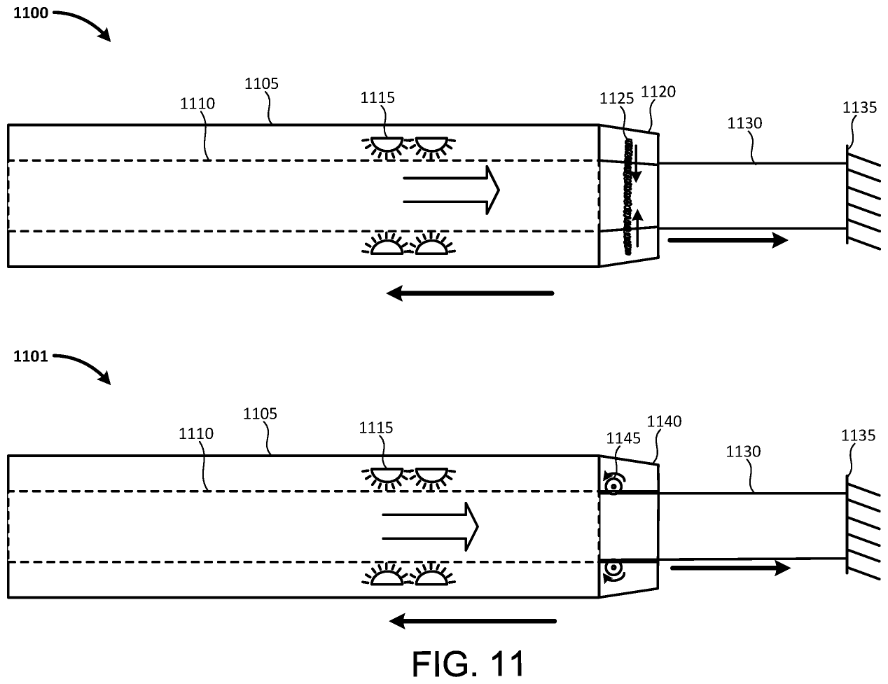

FIG. 11 depicts exemplary closure tensioning modules of exemplary stoma closure tools. A first exemplary stoma closure tool 1100 includes a conduit 1105 defining a lumen 1110. Light-emitting modules 1115 (e.g., at least part of a PTIM) are dispose in the conduit 1105 to emit light into the lumen 1110. The light-emitting modules 1115 may, for example, be configured to (selectively, controllably) initiate photopolymerization of biomaterial as it passes through the lumen 1110 to a distal end (at the right-hand side of the page) of the conduit 1105. Accordingly, the biomaterial may be at least partially solidified as it exits the lumen 1110.

The distal end of the conduit 1105 is provided with a tensioning module 1120. The tensioning module 1120 includes an urging member 1125. The urging member 1125 may, for example, include a spring (e.g., extension spring, (semi-)circular spring band). The urging member 1125 may, for example, apply a radially inward force (as depicted by the diameter of the lumen through the tensioning module 1120 smaller than the diameter of the lumen 1110 in the conduit 1105) such that the tensioning module 1120 applies a radially inward force to the biomaterial as it exits the lumen 1110.

The conduit 1105 may be urged proximally (to the left, as depicted) during deposition to apply a tension to a USCS 1130 formed (e.g., in formation) by the deposited biomaterial. The tensioning module 1120 may, for example, enable an operator and/or (robotic) stoma closure tool system (e.g., as disclosed at least with reference to FIGS. 4A-4C) to maintain a tension against an anchoring point 1135 via the USCS 1130. The anchoring point 1135 (represented schematically) may, for example, be a previously formed anchor of the USCS embedded in and/or against tissue. Applying tension to the USCS 1130 may, for example, advantageously urge at least two tissues together to close a stoma during formation of the USCS. Accordingly, urging the stoma closed may, by way of example and not limitation, be advantageously completed during formation of the USCS in a single process without, for example, requiring separate surgical tools and/or procedures.

A second exemplary stoma closure tool 1101 is provided with a tensioning module 1140. The tensioning module 1140 includes tensioning members 1145. As depicted, the tensioning members 1145 are rotationally coupled to the tensioning module 1140. The tensioning members 1145 may, for example, be provided with a predetermined and/or selectively controllable rotational friction such that the tensioning members 1145 apply tension to the USCS 1130 as it exits the lumen 1110.

Although various embodiments have been described with reference to the figures, other embodiments are possible.

Although an exemplary system has been described with reference to the figures, other implementations may be deployed in other industrial, scientific, medical, commercial, and/or residential applications. For example, various embodiments may provide a minimally invasive stoma closure system configured to deliver and create a biomaterial structure to close one or more stomas in the body (in vivo and in situ) of a living creature. The stoma closure system may include a first lumen in fluid communication with a source of liquid biomaterial. The lumen may, by way of example and not limitation, be defined by a catheter, a hypo tube, a cannula, or some combination thereof. For example, in some embodiments a steerable catheter may be provided with at least the first lumen. In various embodiments multiple lumens may be provided (e.g., for multi-component materials, for (selective) delivery of multiple materials).

In various embodiments a stoma closure system may include a drive module configured to control a rate of flow of the liquid biomaterial through the first lumen. In various embodiments the drive module(s) may be manually and/or automatically controlled. For example, in some embodiments the drive module may include a syringe (e.g., driven by a syringe pump, driven manually, disposed in a handheld dispenser) in fluid communication with the first lumen. In such embodiments the drive module may include an auger dispenser, plunger, or some combination thereof. Various embodiments may be provided, by way of example and not limitation, with a (electronic) drive control. In some embodiments, for example, a drive module(s) may be robotically controlled. The drive module(s) may, for example, induce flow of the liquid biomaterial through the first lumen at a (predetermined) rate of speed. The speed may, for example, be controlled according to a (predetermined) delivery speed profile (e.g., corresponding to time, position of a distal end of the first lumen), according to manual input from an operator (e.g., a physician), or some combination thereof. In some embodiments multiple drive modules may be provided. For example, multiple biomaterials may be selectively dispensed into the first lumen (e.g., selectively controlled by at least one valve). In some embodiments, by way of example and not limitation, multiple biomaterials may be dispensed into multiple lumens.

In various embodiments a stoma closure system may include a phase transition inducement module configured to induce a liquid to solid phase transition in the liquid biomaterial. In various embodiments the phase transition inducement module may include, by way of example and not limitation, an ultraviolet source (e.g., disposed in and/or at a distal end of the first lumen), a mixing mechanism (e.g., to mix multiple components to induce a chemical reaction initiating phase transition of the mixture from liquid to solid), a heat source, or some combination thereof. In various embodiments the biomaterial may include, by way of example and not limitation, photopolymeric material, epoxy, thermoactivated material, enzyme and/or a catalyst reactive material, hydrogel (e.g., alginate, photopolymerized hydrogel, biogel) or some combination thereof.

In various embodiments, when a distal end of a first lumen penetrates through a first tissue and a second tissue which together at least partially define a stoma, the biomaterial may be induced to exit at least one aperture in the distal end of the first lumen. In various embodiments the stoma may include, by way of example and not limitation, a foramen (e.g., in cardiac tissue, in osseous tissue), a septal defect, a perforation, an opening to an appendage (e.g., aneurysm, left atrial appendage, diverticula), a fistula, a conduit (e.g., vasculature), or some combination thereof.

In various embodiments the first lumen may, by way of example and not limitation, be provided with an aperture directly at the end of the lumen, on the side of the lumen substantially at the distal end, having multiple apertures distributed along a distal end region of the first lumen, or some combination thereof. In various embodiments aperture may, by way of example and not limitation, be defined by a curvilinear profile. For example, the aperture may be circular, polygonal, clover-shaped, or some combination thereof. An aperture may, for example, be shaped to advantageously provide desired mechanical, aesthetic, and/or physiological properties to the biomaterial (e.g., during dispensing, after transition to solid form).

The biomaterial may be dispensed in and/or past the second tissue such that the biomaterial forms a solid first anchor in and/or past the second tissue. For example, the solid first anchor may be formed by dispensing liquid material at a first controlled rate relative to a first controlled velocity of the first lumen (e.g., a relatively slow rate of withdrawal, a dwell time sufficient to allow an anchor to be deposited). As depicted in the figure at right, the first anchor may be a substantially spherical structure (e.g., a ‘blob’) formed by increasing the rate of dispensing and/or decreasing and/or pausing a rate of withdrawal from the second tissue.

Once the first anchor is formed in the second tissue, the distal end of the first lumen may be withdrawn from the second tissue such that at least one filament of solid biomaterial, continuous with the first anchor, is formed across the stoma. For example, the at least one filament may be formed by dispensing the liquid biomaterial at a second controlled rate relative to a second controlled velocity of the first lumen. In various embodiments the second controlled rate of dispensing of the liquid biomaterial may be less than the first controlled rate and/or the second velocity of the first lumen may be greater than the first velocity. Accordingly, the filament may be mechanically connected by continuous material formation with the first anchor. The first anchor may, for example, provide a robust connection point in the second tissue for the filament.

As the distal end of the first lumen is withdrawn into and/or through the second tissue, a second anchor of solid biomaterial may be formed continuously with the at least one filament and the second anchor. For example, the second anchor may be formed by dispensing the liquid biomaterial at a third controlled rate relative to a third controlled velocity of the first lumen. In various embodiments the third controlled rate of dispensing may be greater than the second controlled rate and/or the third velocity may be greater than the second velocity. Accordingly, the first and second anchors connected by the filament by continuous material formation may provide a unitary closure of solid biomaterial formed in situ and in vivo across the stoma in a minimally invasive operation. For example, the unitary closure may be adapted (e.g., in real time) to the unique conditions and/or geometry of the patient.

In various embodiments multiple unitary closures may be formed across one or more stomata by repetition of the process. Unitary closures may, by way of example and not limitation, mechanically close the stoma (e.g., by bringing and/or retaining the first and second tissues against each other), provide a scaffold for regenerative remodeling (e.g., by the patient's body, by delivering of therapeutic growth factors and/or cells), constrain the stoma to a desired level of closure (e.g., partially open), or some combination thereof.

In various embodiments a rate of dispensing and/or a velocity of at least the first lumen may be controlled manually and/or robotically. For example, the first lumen may be defined by a steerable catheter. The catheter may be handheld, steered directly by a human operator, robotically mounted, steered by electronic controls, or some combination thereof. Accordingly, in various embodiments the unitary closure(s) may be free form, predetermined (e.g., according to previous imagery and/or modeling of the stoma, the patient's body, and/or the unitary closure(s)), or some combination thereof.

In various embodiments, formation of the unitary closures (e.g., position and/or motion of the lumen, dispensing of the biomaterial(s)) may be guided by concurrent imaging (e.g., ultrasound, radiography, fluoroscopy, magnetic resonance). For example, in some embodiments biomaterial may be provided with imaging component(s) such as, by way of example and not limitation, fluorescent markers, magnetically susceptible components, radiopaque material, or some combination thereof.

In various embodiments a tip of a conduit (e.g., as disclosed at least with reference to FIG. 9) may be selectively controllable. For example, a size and/or geometry of the tip may be adjustable (e.g., robotically, in response to operator inputs). The tip may, for example, be operated into a first geometry and/or size to form an anchor and operated into a second geometry and/or size to form a bridge(s). Valves may, for example, open and/or close individual lumens in a tip. For example, a lumen may be opened to form an anchor and closed while bridges are being formed. Accordingly, complex anchor and/or bridge geometries (e.g., combinations of the exemplary USCS geometries disclosed at least with reference to FIG. 10) may be advantageously formed.

In various embodiments, some bypass circuits implementations may be controlled in response to signals from analog or digital components, which may be discrete, integrated, or a combination of each. Some embodiments may include programmed, programmable devices, or some combination thereof (e.g., PLAs, PLDs, ASICs, microcontroller, microprocessor), and may include one or more data stores (e.g., cell, register, block, page) that provide single or multi-level digital data storage capability, and which may be volatile, non-volatile, or some combination thereof. Some control functions may be implemented in hardware, software, firmware, or a combination of any of them.

Computer program products may contain a set of instructions that, when executed by a processor device, cause the processor to perform prescribed functions. These functions may be performed in conjunction with controlled devices in operable communication with the processor. Computer program products, which may include software, may be stored in a data store tangibly embedded on a storage medium, such as an electronic, magnetic, or rotating storage device, and may be fixed or removable (e.g., hard disk, floppy disk, thumb drive, CD, DVD).

Although an example of a system, which may be portable, has been described with reference to the above figures, other implementations may be deployed in other processing applications, such as desktop and networked environments.

Temporary auxiliary energy inputs may be received, for example, from chargeable or single use batteries, which may enable use in portable or remote applications. Some embodiments may operate with other DC voltage sources, such as a 9V (nominal) battery, for example. Alternating current (AC) inputs, which may be provided, for example from a 50/60 Hz power port, or from a portable electric generator, may be received via a rectifier and appropriate scaling. Provision for AC (e.g., sine wave, square wave, triangular wave) inputs may include a line frequency transformer to provide voltage step-up, voltage step-down, and/or isolation.

Although particular features of an architecture have been described, other features may be incorporated to improve performance. For example, caching (e.g., L1, L2, . . . ) techniques may be used. Random access memory may be included, for example, to provide scratch pad memory and or to load executable code or parameter information stored for use during runtime operations. Other hardware and software may be provided to perform operations, such as network or other communications using one or more protocols, wireless (e.g., infrared) communications, stored operational energy and power supplies (e.g., batteries), switching and/or linear power supply circuits, software maintenance (e.g., self-test, upgrades), and the like. One or more communication interfaces may be provided in support of data storage and related operations.

Some systems may be implemented as a computer system that can be used with various implementations. For example, various implementations may include digital circuitry, analog circuitry, computer hardware, firmware, software, or combinations thereof. Apparatus can be implemented in a computer program product tangibly embodied in an information carrier, e.g., in a machine-readable storage device, for execution by a programmable processor; and methods can be performed by a programmable processor executing a program of instructions to perform functions of various embodiments by operating on input data and generating an output. Various embodiments can be implemented advantageously in one or more computer programs that are executable on a programmable system including at least one programmable processor coupled to receive data and instructions from, and to transmit data and instructions to, a data storage system, at least one input device, and/or at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, which may include a single processor or one of multiple processors of any kind of computer. Generally, a processor will receive instructions and data from a read-only memory or a random-access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including, by way of example, semiconductor memory devices, such as EPROM, EEPROM, and flash memory devices; magnetic disks, such as internal hard disks and removable disks; magneto-optical disks; and CD-ROM and DVD-ROM disks. The processor and the memory can be supplemented by, or incorporated in, ASICs (application-specific integrated circuits).

In some implementations, each system may be programmed with the same or similar information and/or initialized with substantially identical information stored in volatile and/or non-volatile memory. For example, one data interface may be configured to perform auto configuration, auto download, and/or auto update functions when coupled to an appropriate host device, such as a desktop computer or a server.

In some implementations, one or more user-interface features may be custom configured to perform specific functions. Various embodiments may be implemented in a computer system that includes a graphical user interface and/or an Internet browser. To provide for interaction with a user, some implementations may be implemented on a computer having a display device, such as a CRT (cathode ray tube) or LCD (liquid crystal display) monitor for displaying information to the user, a keyboard, and a pointing device, such as a mouse or a trackball by which the user can provide input to the computer.

In various implementations, the system may communicate using suitable communication methods, equipment, and techniques. For example, the system may communicate with compatible devices (e.g., devices capable of transferring data to and/or from the system) using point-to-point communication in which a message is transported directly from the source to the receiver over a dedicated physical link (e.g., fiber optic link, point-to-point wiring, daisy-chain). The components of the system may exchange information by any form or medium of analog or digital data communication, including packet-based messages on a communication network. Examples of communication networks include, e.g., a LAN (local area network), a WAN (wide area network), MAN (metropolitan area network), wireless and/or optical networks, the computers and networks forming the Internet, or some combination thereof. Other implementations may transport messages by broadcasting to all or substantially all devices that are coupled together by a communication network, for example, by using omni-directional radio frequency (RF) signals. Still other implementations may transport messages characterized by high directivity, such as RF signals transmitted using directional (i.e., narrow beam) antennas or infrared signals that may optionally be used with focusing optics. Still other implementations are possible using appropriate interfaces and protocols such as, by way of example and not intended to be limiting, USB 2.0, Firewire, ATA/IDE, RS-232, RS-422, RS-485, 802.11 a/b/g, Wi-Fi, Ethernet, IrDA, FDDI (fiber distributed data interface), token-ring networks, multiplexing techniques based on frequency, time, or code division, or some combination thereof. Some implementations may optionally incorporate features such as error checking and correction (ECC) for data integrity, or security measures, such as encryption (e.g., WEP) and password protection.

In various embodiments, the computer system may include Internet of Things (IoT) devices. IoT devices may include objects embedded with electronics, software, sensors, actuators, and network connectivity which enable these objects to collect and exchange data. IoT devices may be in-use with wired or wireless devices by sending data through an interface to another device. IoT devices may collect useful data and then autonomously flow the data between other devices.

Various examples of modules may be implemented using circuitry, including various electronic hardware. By way of example and not limitation, the hardware may include transistors, resistors, capacitors, switches, integrated circuits, other modules, or some combination thereof. In various examples, the modules may include analog logic, digital logic, discrete components, traces and/or memory circuits fabricated on a silicon substrate including various integrated circuits (e.g., FPGAs, ASICs), or some combination thereof. In some embodiments, the module(s) may involve execution of preprogrammed instructions, software executed by a processor, or some combination thereof. For example, various modules may involve both hardware and software.

A number of implementations have been described. Nevertheless, it will be understood that various modifications may be made. For example, advantageous results may be achieved if the steps of the disclosed techniques were performed in a different sequence, or if components of the disclosed systems were combined in a different manner, or if the components were supplemented with other components. Accordingly, other implementations are contemplated within the scope of the following claims.

What is claimed is:

1. A method of stoma closure, the method comprising:
insert, with a stoma closure tool comprising a drive module, a phase transition inducement module (PTIM), a conduit that defines a lumen, and a tensioning module, a distal end of the conduit through a first tissue and into a second tissue that together at least partially define a stoma;
control, by the drive module, a flow rate of a fluid biomaterial through the lumen and discharged at the distal end of the conduit; and,
induce, by the PTIM a fluid to solid phase transition in the fluid biomaterial such that the discharged biomaterial forms at least one continuous structure extending directly across the stoma between a proximal anchor in the first tissue and a distal anchor in the second tissue,
wherein after formation of the distal anchor, the tensioning module applies tension to the at least one continuous structure such that the distal anchor urges the second tissue and the first tissue towards one another.

2. The method of claim 1, wherein the fluid biomaterial comprises a photopolymer and the PTIM comprises a selectively activated light source.

3. The method of claim 1, wherein:
the fluid biomaterial comprises a first component and a second component,
mixing the first component and the second component induces the phase transition from fluid to solid, and
the PTIM comprises a mechanism configured to mix the first component and the second component.

4. The method of claim 1, the method further comprising apply tension to the at least one continuous structure until the proximal anchor is formed.

5. The method of claim 1, the method further comprising forming a spacing element into the at least one continuous structure between the first tissue and the second tissue.

6. The method of claim 1, wherein the conduit defines a plurality of lumens such that the at least one continuous structure comprises a corresponding plurality of filaments connecting the distal anchor and the proximal anchor.

7. A method of stoma closure, the method comprising:
insert, with a stoma closure tool comprising a drive module, a phase transition inducement module (PTIM), and a conduit that defines a lumen, a distal end of the conduit through a first tissue and into a second tissue that together at least partially define a stoma;
control, by the drive module, a flow rate of a fluid biomaterial through the lumen and discharged at the distal end of the conduit; and,
induce, by the PTIM a fluid to solid phase transition in the fluid biomaterial such that the discharged biomaterial forms at least one continuous structure extending directly across the stoma between a proximal anchor in the first tissue and a distal anchor in the second tissue.

8. The method of claim 7, wherein the fluid biomaterial comprises a liquid.

9. The method of claim 7, wherein the fluid biomaterial comprises a photopolymer.

10. The method of claim 9, wherein the PTIM comprises a selectively activated light source.

11. The method of claim 7, wherein the fluid biomaterial comprises a first component and a second component, and wherein mixing the first component and the second component induces the phase transition from fluid to solid.

12. The method of claim 11, wherein the PTIM comprises a mechanism configured to mix the first component and the second component.

13. The method of claim 7, wherein the stoma comprises a distension in a wall defining an internal cavity of an organism.

14. The method of claim 7, wherein the stoma comprises a passageway into at least one internal cavity of an organism.

15. The method of claim 14, wherein the passageway is defined by the first tissue and the second tissue, and the first tissue and the second tissue overlap.

16. The method of claim 7, wherein:
the stoma closure tool further comprises a tensioning module, and,
the method further comprises, after formation of the distal anchor, apply tension, by the tensioning module, to the at least one continuous structure such that the distal anchor urges the second tissue and the first tissue towards one another.

17. The method of claim 16, the method further comprising apply tension to the at least one continuous structure until the proximal anchor is formed.

18. The method of claim 7, the method further comprising forming a spacing element into the at least one continuous structure between the first tissue and the second tissue.

19. The method of claim 7, wherein the conduit defines a plurality of lumens such that the at least one continuous structure comprises a corresponding plurality of filaments connecting the distal anchor and the proximal anchor.

20. The method of claim 7, wherein:
the stoma closure tool further comprises a cross-section control module configured to selectively control a geometry of the lumen at the distal end of the conduit, and,
the method further comprises operate the cross-section control module, after the distal anchor is formed, to transition the geometry of the lumen from a first configuration to a second configuration such that a cross-section of the biomaterial dispensed is correspondingly transitioned from a first cross-sectional geometry to a second cross-sectional geometry.
